(19) United States
(12) Patent Application Publication
(10) Pub. No.: US 2020/0157208 A1
(43) Pub. Date: May 21, 2020

(54) COMBINATION OF AN ANTIBODY THAT BINDS TO THE P19 SUBUNIT OF HUMAN IL-23 AND A HYALURONIDASE ENZYME

(71) Applicant: Eli Lilly and Company, ndianapolis, IN (US)

(72) Inventor: Philip Barrington, High Wycombe (GB)

(21) Appl. No.: 16/686,921

(22) Filed: Nov. 18, 2019

Related U.S. Application Data
(60) Provisional application No. 62/769,278, filed on Nov. 19, 2018.

(51) Int. Cl. C07K 16/24; C12N 9/26

(52) U.S. Cl. CPC C07K 16/244; C12Y 302/01035; C12N 9/2474

(57) ABSTRACT

The present invention generally relates to the combination of an antibody that binds to the p19 subunit of human IL-23 and a hyaluronidase enzyme and uses thereof.

The present invention is in the field of medicine. More specifically, the present invention relates to treatments and treatment regimen with an antibody that binds to human interleukin-23 (IL-23)(an anti-IL-23p19 antibody) by subcutaneous injection.

Human IL-23, a member of the interleukin-12 (IL-12) family of cytokines, is a heterodimeric protein composed of two subunits: the p40 subunit, which is shared by IL-12, and the p19 subunit, which is specific to IL-23. IL-23 is produced by antigen-presenting cells, such as dendritic cells and macrophages and is critically involved in the maintenance and amplification of T helper 17 (Th17) cells. Stimulation of these cells with IL-23 induces a unique inflammatory signature that includes interleukin-17A, interleukin-17F, interleukin-6, granulocyte-macrophage-colony stimulating factor, tumor necrosis factor α, chemokine ligand 20, chemokine ligand 22, and IL-23 receptor. In addition to Th17 cells, many innate immune cells respond to IL-23 and are important both in resistance to infection and in mediating pathology in many autoimmune/inflammatory diseases including ulcerative colitis (UC) and Crohn's Disease (CD). Treatment of a number of conditions with IL-23 targeted therapy is being pursued by several companies. The first such biologic to demonstrate clinical benefit in autoimmune disease was ustekinumab, which is a Food and Drug Administration-approved monoclonal antibody for the treatment of psoriasis and psoriatic arthritis and CD. Ustekinumab binds the common p40 subunit of IL-12 and IL-23; therefore, it targets both cytokines, rather than IL-23 specifically. Blockade of the IL-12 pathway may prevent Th1 cell-induced interferon blockade of Th17 cell development, thus potentially limiting the clinical activity of p40 targeting antibodies.

Agents targeting the IL-23 p19 subunit (i.e. bind to human IL-23 but do not bind to human IL-12) have demonstrated clinical activity in psoriasis and CD (Kopp T et al., Nature, Volume 521, No. 7551, pages 222-226, 2015; Sands BE et al., Journal of Crohn's and Colitis, Volume 9, Issue Supplement 1, ppS15-S16, 2015).

Guselkumab, CAS Registry No. 1350289-85-8, is a fully human IgG₁ lambda monoclonal antibody that binds to the p19 subunit of human IL-23 that has been approved for the treatment of plaque psoriasis. The antibody and methods of making same are described in U.S. Pat. No. 7,935,344. 100 mg of guselkumab is formulated in L-histidine (0.6 mg), L-histidine monohydrochloride monohydrate (1.5 mg), polysorbate 80 (0.5 mg), sucrose (79 mg) and water for injection at pH 5.8 in a pre-filled single dose syringe that delivers 1 mL of solution (see FDA label for Tremfya®) subcutaneously.

Tildrakizumab, CAS Registry No. 1326244-10-3, is a humanized, IgG1 kappa monoclonal antibody targeting the p19 subunit of human IL-23 that has approved for the treatment of moderate to severe plaque psoriasis. The antibody and methods of making same are described in U.S. Pat. No. 8,293,883. 100 mg of tildrakizumab is formulated in L-histidine (0.495 mg), L-histidine hydrochloride monohydrate (1.42 mg), polysorbate 80 (0.5 mg), sucrose (70.0 mg), and water for injection (USP with a pH of 5.7-6.3) in a pre-filled single dose syringe that delivers 1 mL of solution (see FDA label for Ilumya®) subcutaneously.

Risankizumab, CAS Registry No. 1612838-76-2, is a humanized, IgG1 kappa monoclonal antibody targeting the p19 subunit of human IL-23. The antibody and methods of making same are described in U.S. Pat. No. 8,778,346. Risankizumab is being evaluated for the treatment of psoriasis, CD, UC and psoriatic arthritis

Brazikumab, CAS Registry No. 1610353-18-8, is a humanized, IgG₂-lambda monoclonal antibody targeting the p19 subunit of human IL-23. The antibody and methods of making same are described in U.S. Pat. No. 8,722,033. Brazikumab is being evaluated for the treatment CD and UC.

Mirikizumab, CAS Registry No. 1884201-71-1, is a humanized immunoglobulin (Ig) G4-variant monoclonal antibody targeting the p19 subunit of human IL-23. Mirikizumab has been evaluated in a Phase I study of healthy volunteers and in subjects with plaque psoriasis (Study I6T-MC-AMAA; Maari C et al., EADV 2016). Mirikizumab is being evaluated for the treatment of patients with moderate to severe plaque psoriasis, UC and CD.

While the pharmaceutical use of antibodies is now reasonably common, in many instances such antibodies are injected via the intravenous (IV) route. Unfortunately the amount of antibody that can be injected via the intravenous route is limited by the physico-chemical properties of the antibody, in particularly by its solubility and stability in a suitable liquid formulation and by the volume of the infusion fluid. Alternative administration pathways are subcutaneous or intramuscular injection. These injection pathways require high protein concentration in the final solution to be injected (Shire, S J, Shahrokh, Z et al., “Challenges in the development of high protein concentration formulations”, J Pharm. Sci., 93(6), 1390-1402, 2004; Roskos, L K, Davis C G et al., “The clinical pharmacology of therapeutic antibodies”, Drug Development Research, 61(3), 108-120, 2004). In order to increase the volume, and thereby the therapeutic dose, which can be safely and comfortably administered subcutaneously it has been proposed to use glycosaminoglycanase enzyme(s) in order to increase the interstitial space into which the antibody formulation can be injected (WO 2006/091871 A1).

The advantage of subcutaneous injection is that it allows the medical practitioner to perform it in a reasonably short intervention with the patient. Moreover, the patient can be trained to self-administer the subcutaneous injection. Such self-administration is particularly useful during maintenance dosing because no hospital care is needed (reduced medical resource utilization). Typically injections via the subcutaneous route are limited to approximately 2 ml. For patients requiring multiple doses, several unit dose formulations may be injected at multiple sites of the body surface.

A disadvantage of subcutaneous injection is the possibility of pain at the injection site, even after the needle has been removed. This may be influenced by components of the protein solution, such as the protein itself, the sort of buffer molecules and the osmolarity. Injection site pain may have a significant influence on patient compliance of the respective therapy.

It is anticipated that a number of the medical conditions being evaluated for treatment by antibodies that bind to the p19 subunit of human IL-23 may require high doses for effective treatment thereof. For the purposes of patient compliance, it is preferable to minimize the number of doses administered to the patient to achieve effective treatment of a condition. Treatments and treatment regimen that facilitate administration of anti-IL-23p19 antibodies at high doses by subcutaneous injection are thus highly desirable in instances where high doses of the antibody are required for effective treatment of a condition.

Accordingly, there remains a need to provide treatments and treatment regimen that facilitate administration of anti-IL-23p19 antibodies at high doses by subcutaneous injection and minimise injection site pain.

In a first aspect of the present invention, there is provided a method of treating an IL-23 related disorder wherein the method comprises administering in simultaneous, separate or sequential combination to a patient in need thereof, effective amounts of mirikizumab and a hyaluronidase enzyme.

It has been surprisingly found that administration of mirikizumab with a hyaluronidase enzyme results in a reduction of injection site pain when compared to administration of a mirikizumab alone. As illustrated in the Examples, a formulation comprising mirikizumab and a hyaluronidase enzyme administered to human patients has a statistically significant lower pain VAS score than an equivalent formulation comprising mirikizumab but not comprising a hyaluronidase enzyme.

A hyaluronidase enzyme transiently increases the dispersion and absorption of co-administered agents by depolymerizing hyaluronan in the subcutaneous space. Hyaluronan is a repeating polymer of N-acetyl glucosamine and glucuronic acid that contributes to the soluble gel-like component of the extracellular matrix of the skin. Depolymerisation of hyaluronan by hyaluronidase is accomplished by enzymatic hydrolysis of the covalent bonds between the disaccharide units of the polymer, and results in a transient reduction in viscosity of the gel-like phase of the extracellular matrix and an increased hydraulic conductance that allows larger volumes to be administered into the subcutaneous tissue. The increased hydraulic conductance induced by hyaluronidase enzyme through reduced interstitial viscosity enables greater dispersion, potentially increasing the systemic bioavailability of therapeutic drugs administered subcutaneously.

Mirikizumab and the hyaluronidase enzyme may be provided for administration in form of one single combined formulation or alternatively in form of two separate formulations which can be mixed just prior to the subcutaneous injection. Alternatively mirikizumab and the hyaluronidase enzyme can be administered as separate injections at different sites of the body, preferably at sites which are immediately adjacent to each other. It is also possible to inject the therapeutic agents present in the formulation in accordance with the present invention as consecutive injections, e.g. first the hyaluronidase enzyme followed by the injection of mirikizumab formulation. These injections can also be performed in the reverse order, viz. by first injecting the mirikizumab formulation followed by injecting the hyaluronidase enzyme.

The hyaluronidase enzyme may be considered to be a further excipient in a mirikizumab formulation. The hyaluronidase enzyme may be added to the mirikizumab formulation at the time of manufacturing the mirikizumab formulation or may be added shortly before the injection. Alternatively, the hyaluronidase enzyme may be provided as a separate injection. In the latter case the hyaluronidase enzyme may be provided in a separate vial either in lyophilized form which must be reconstituted with suitable diluents before the subcutaneous injection takes place, or may be provided as a liquid formulation by the manufacturer. The mirikizumab and the hyaluronidase enzymes may be procured as separate entities or may also be provided as kits comprising both injection components and suitable instructions for their subcutaneous administration. Suitable instructions for the reconstitution and/or administration of one or both of the formulations may also be provided.

In a further embodiment of the present invention, the hyaluronidase enzyme is recombinant human hyaluronidase PH20 (rHuPH20)(CAS-Registry No. 757971-58-7). The preferred hyaluronidase enzyme is a hyaluronidase glycoprotein comprising 447 amino acids with an approximate molecular weight of 61,000 Daltons as described in U.S. Pat. No. 7,767,429. rHuPH20 is the active ingredient in the commercial product HYLENEX® (recombinant (hyaluronidase human injection), approved by the US Food and Drug Administration (FDA) in 2005.

In a still further embodiment of the present invention, the effective amount of mirikizumab is 50-5000 mg.

Further preferably, the effective amount of mirikizumab is 50 mg, 100 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg.

Still further preferably, the effective amount of mirikizumab is 100 mg. For example, the 100 mg of mirikizumab may be administered as one injection of 100 mg in 1 mL of formulated injection solution.

Alternatively preferably, the effective amount of mirikizumab is 125 mg. For example, the 125 mg of mirikizumab may be administered as one injection of 125 mg in 1 mL of the formulated injection solution.

Alternatively preferably, the effective amount of mirikizumab is 200 mg. For example, the 200 mg of mirikizumab may be administered SC as two injections of 100 mg in 1 mL of formulated injection solution or administered SC as one injection of 200 mg in 2 mL of formulated injection solution.

Alternatively preferably, the effective amount of mirikizumab is 250 mg. For example, the 250 mg of mirikizumab may be administered SC as two injections of 125 mg in 1 mL of formulated injection solution or administered SC one injection of 250 mg in 2 mL of formulated injection solution.

Alternatively preferably, the effective amount of mirikizumab is 600 mg.

Alternatively preferably, the effective amount of mirikizumab is 1000 mg.

Alternatively preferably, the effective amount of mirikizumab is 1200 mg.

Alternatively preferably, the effective amount of mirikizumab is 2400 mg.

The effective amount of hyaluronidase enzyme is related to the injection volume. Accordingly, in a still further embodiment of the present invention, the effective amount of hyaluronidase enzyme is 1000-3000 U per mL of formulated injection solution.

Preferably, the effective amount of hyaluronidase enzyme is 1500-2500 U per mL of formulated injection solution.

Still further preferably, the effective amount of hyaluronidase enzyme is 2000 U per mL of formulated injection solution.

The maximum effective amount of rHuPH20 is 50,000 U. Accordingly, when the effective amount of hyaluronidase enzyme is 2000 U per mL of formulated injection solution, the maximum volume of formulated injection solution is 25 mL. Increasing the amount of rHuPH20 beyond 50,000 U is expected to yield little additional benefit.

In a still further embodiment of the present invention, the IL-23 related disorder is psoriasis, psoriatic arthritis, UC, CD or ankylosing spondylitis.

In a preferred embodiment of the present invention, mirikizumab and the hyaluronidase enzyme are co-formulated for simultaneous administration by subcutaneous injection.

In a further aspect of the present invention, there is provided mirikizumab for use in the treatment of an IL-23 related disorder, wherein mirikizumab is administered in simultaneous, separate or sequential combination with a hyaluronidase enzyme and wherein administration of mirikizumab and the hyaluronidase enzyme is by subcutaneous injection.

In a still further embodiment of the present invention, the hyaluronidase enzyme is recombinant human hyaluronidase enzyme (CAS-Registry No. 757971-58-7).

In a further embodiment of the present invention, the amount of mirikizumab administered is 50-5000 mg.

Preferably, the amount of mirikizumab administered is 50 mg, 100 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg.

Still further preferably, the amount of mirikizumab administered is 100 mg. For example, the 100 mg of mirikizumab may be administered as one injection of 100 mg in 1 mL of formulated injection solution

Alternatively preferably, the amount of mirikizumab administered is 125 mg. For example, the 125 mg of mirikizumab may be administered as one injection of 125 mg in 1 mL of formulated injection solution.

Alternatively preferably, the amount of mirikizumab administered is 200 mg. For example, the 200 mg of mirikizumab may be administered SC as two injections of 100 mg in 1 mL of formulated injection solution or administered as one injection of 200 mg in 2 mL of formulated injection solution.

Alternatively preferably, the amount of mirikizumab administered is 250 mg. For example, the 250 mg of mirikizumab may be administered SC as two injections of 125 mg in 1 mL of formulated injection solution or administered SC as one injection of 250 mg in 2 mL of formulated injection solution.

Alternatively preferably, the amount of mirikizumab administered is 600 mg.

Alternatively preferably, the amount of mirikizumab administered is 1000 mg.

Alternatively preferably, the amount of mirikizumab administered is 1200 mg.

Alternatively preferably, the amount of mirikizumab administered is 2400 mg.

The amount of hyaluronidase enzyme to be administered is related to the injection volume.

Accordingly, in a still further embodiment of the present invention, the amount of hyaluronidase enzyme administered is 1000-3000 U per mL of formulated injection solution.

Preferably, the amount of hyaluronidase enzyme administered is 1500-2500 U per mL of formulated injection solution.

Still further preferably, the amount of hyaluronidase enzyme administered is 2000 U per mL of formulated injection solution.

The maximum amount of rHuPH20 is 50,000 U. Accordingly, when the amount of hyaluronidase enzyme is 2000 U per mL of formulated injection solution, the maximum volume of formulated injection solution is 25 mL. Increasing the amount of rHuPH20 beyond 50,000 U is expected to yield little additional benefit.

In a further embodiment of the present invention, the IL-23 related disorder is psoriasis, psoriatic arthritis, UC, CD or ankylosing spondylitis.

In a preferred embodiment of the present invention, mirikizumab and the hyaluronidase enzyme are co-formulated for simultaneous administration by subcutaneous injection.

In a further aspect of the present invention, there is provided a combination comprising mirikizumab and a hyaluronidase enzyme, wherein the combination is administered by subcutaneous injection.

In a further embodiment of the present invention, the hyaluronidase enzyme is recombinant human hyaluronidase enzyme (CAS-Registry No. 757971-58-7).

In a still further embodiment of the present invention, the combination comprises 50-5000 mg of mirikizumab.

Preferably, the combination comprises 50 mg, 100 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg of mirikizumab.

Further preferably, the combination comprises 100 mg of mirikizumab. For example, the 100 mg of mirikizumab may be administered as one injection of 100 mg in 1 mL of formulated injection solution.

Alternatively preferably, the combination comprises 125 mg of mirikizumab. For example, the 125 mg of mirikizumab may be administered as one injection of 125 mg in 1 mL of formulated injection solution.

Alternatively preferably, the combination comprises 200 mg of mirikizumab. For example, the 200 mg of mirikizumab may be administered SC as two injections of 100 mg in 1 mL of formulated injection solution or administered SC as one injection of 200 mg in 2 mL of formulated injection solution.

Alternatively preferably, the combination comprises 250 mg of mirikizumab. For example, the 250 mg of mirikizumab may be administered SC as two injections of 125 mg in 1 mL of injection solution or one injection of 250 mg in 2 mL of injection solution.

Alternatively preferably, the combination comprises 600 mg of mirikizumab.

Alternatively preferably, the combination comprises 1000 mg of mirikizumab.

Alternatively preferably, the combination comprises 1200 mg of mirikizumab.

Alternatively preferably, the combination comprises 2400 mg of mirikizumab.

The amount of hyaluronidase enzyme to be administered is related to the injection volume.

Accordingly, in a still further embodiment of the invention, the combination comprises 1000-3000 U hyaluronidase enzyme per mL of formulated injection solution.

Preferably, the combination comprises 1500-2500 U of hyaluronidase enzyme 1500-2500 U per mL of formulated injection solution.

Still further preferably, the combination comprises 2000 U of hyaluronidase enzyme per mL of formulated injection solution.

The maximum amount of rHuPH20 is 50,000 U. Accordingly, when the amount of hyaluronidase enzyme is 2000 U per mL of formulated injection solution, the maximum volume of formulated injection solution is 25 mL. Increasing the amount of rHuPH20 beyond 50,000 U is expected to yield little additional benefit.

In a still further aspect of the present invention, there is provided a pharmaceutical formulation for subcutaneous injection comprising 100-2400 mg of mirikizumab, and 1500-3000 U of recombinant human hyaluronidase enzyme (rHuPH20)(CAS-Registry No. 757971-58-7) per mL of injection solution.

In a further embodiment of the present invention, the pharmaceutical formulation comprises 100-2400 mg of mirikizumab, and 2000 U of recombinant human hyaluronidase enzyme (rHuPH20)(CAS-Registry No. 757971-58-7) per mL of formulated injection solution.

Preferably, the pharmaceutical formulation comprises 100 mg of mirikizumab, and 2000 U of rHuPH20 in 1 mL of formulated injection solution.

Alternatively preferably, the pharmaceutical formulation comprises 150 mg of mirikizumab, and 2000 U of rHuPH20 in 1 mL of formulated injection solution.

Alternatively preferably, the pharmaceutical formulation comprises 200 mg of mirikizumab, and 4000 U of rHuPH20 in 2 mL of formulated injection solution.

Alternatively preferably, the pharmaceutical formulation comprises 250 mg of mirikizumab, and 4000 U of rHuPH20 in 2 mL of formulated injection solution.

Alternatively preferably, the pharmaceutical formulation comprises 600 mg of mirikizumab, and 12,000 U of rHuPH20 in 6 mL of formulated injection solution.

Alternatively preferably, the pharmaceutical formulation comprises 1200 mg of mirikizumab and 16,200 U of rHuPH20 in 8.1 mL of formulated injection solution.

Alternatively preferably, the pharmaceutical formulation comprises 2400 mg of mirikizumab and 39,200 U of rHuPH20 in 19.5 mL of formulated injection solution.

In a further aspect of the present invention, there is provided a kit comprising a first pharmaceutical formulation comprising 50-5000 mg of mirikizumab and a second pharmaceutical formulation comprising a recombinant human hyaluronidase enzyme (CAS-Registry No. 757971-58-7), wherein the kit further comprises instructions for combining the first and second formulations for co-administration such that the co-formulated formulation is suitable for subcutaneous injection and comprises 50-5000 mg of mirikizumab and 1000-3000 U of rHuPH20 per mL of formulated injection solution.

As used herein, the terms “treating,” “treat,” or “treatment,” refer to restraining, slowing, lessening, reducing, or reversing the progression or severity of an existing symptom, disorder, condition, or disease, or ameliorating clinical symptoms and/or signs of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Those in need of treatment include those already with the disease.

As used herein, the term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a human IL-23. For example, antibody fragments capable of binding to IL-12/23 or portions thereof, including, but not limited to, Fab (e.g. by papain digestion), Fab′ (e.g.; by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and re-aggregation), Fv or scFv (e.g. by molecular biology techniques) fragments, are encompassed by the present invention (see, e.g. Colligan et al., Current Protocols in Immunology, John Wiley & Sons, NY, N.Y., (1994-2001)).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

As used herein “an antibody that binds to the p19 subunit of human IL-23” refers to an antibody that binds to the p19 subunit of human IL-23 but does not bind to the p40 subunit of human IL-23. An “antibody that binds to the p19 subunit of human IL-23” thus binds to human IL-23 but does not bind to human IL-12.

As used herein, the term “patient” refers to a mammal, preferably a human.

As used herein, the term “kit” refers to a package comprising at least two separate containers, wherein a first container contains mirikizumab and a second container contains a hyaluronidase enzyme. A “kit” may also include instructions to administer all or a portion of the contents of these first and second containers to a cancer patient.

As used herein, the term “effective amount” or “effective dose” refers to the amount of mirikizumab and/or a hyaluronidase enzyme or pharmaceutical composition comprising mirikizumab and/or a hyaluronidase enzyme that will elicit the biological or medical response of or desired therapeutic effect on a tissue, system, animal, mammal or human that is being sought by the researcher, medical doctor, or other clinician. An effective amount of the antibody may vary according to factors such as the disease state, age, sex, and weight of the individual, and the ability of the antibody to elicit a desired response in the individual. An effective amount is also one in which any toxic or detrimental effect of the antibody is outweighed by the therapeutically beneficial effects.

As used herein, the phrase “an IL-23 related disorder” refers to a condition in which IL-23 activity contributes to the disease and typically where IL-23 is abnormally expressed. An IL-23-associated disorder includes diseases and disorders of the immune system, such as autoimmune disorders and inflammatory disorders. Such conditions include, but are not limited to, rheumatoid arthritis (RA), systemic lupus erythematosus (SLE), scleroderma, Sjogren's syndrome, multiple sclerosis, psoriasis, psoriatic arthritis, inflammatory bowel disease (e.g., UC and CD), pulmonary inflammation, asthma, idiopathic thrombocytopenic purpura (ITP) and ankylosing spondylitis.

As used herein, the phrase “in combination with” refers to the administration of mirikizumab with a hyaluronidase enzyme simultaneously. As used herein, the phrase “in combination with” also refers to the administration of mirikizumab with a hyaluronidase enzyme sequentially in any order. As used herein, the phrase “in combination with” also refers to the administration of mirikizumab with a hyaluronidase enzyme in any combination thereof. Mirikizumab can be administered prior to administration of a hyaluronidase enzyme. Mirikizumab can be administered at the same time as administration of a hyaluronidase enzyme. Mirikizumab can be administered subsequent to administration of a hyaluronidase enzyme. Mirikizumab can be administered prior to, at the same time as, or subsequent to administration of hyaluronidase enzyme, or in some combination thereof.

As used herein, the phrase “injection site pain” refers to pain attributable to injection of a liquid formulation subcutaneously and localized to the site of the injection. Pain may be evaluated using any type of pain assessment known in the art, including, for example, visual analog scales (VAS), qualitative assessments of pain, or needle pain assessments. For example, subject-perceived injection site pain may be assessed using the Pain Visual Analog Scale (VAS). A VAS is a measurement instrument that measures pain as it ranges across a continuum of values, e.g., from none to an extreme amount of pain. Operationally a VAS is a horizontal line, about 100 mm in length, anchored by numerical and/or word descriptors, e.g., 0 or 10, or “no pain” or “excruciating pain,” optionally with additional word or numeric descriptors between the extremes, e.g., mild, moderate, and severe; or 1 through 9) (see, e.g., Lee J S, et al. (2000) AcadEmerg Med 7:550, or Singer and Thods (1998) Academic Emergency Medicine, 5:1007). Pain may be assessed at a single time or at various times following administration of a formulation such as, for example, immediately after injection, at about 1, 2, 3, 4, 5, 6, 7, 8, 9, 10, 15, 20, 25, 30, 35, 40, or 45 minutes after injection.

### BRIEF DESCRIPTION OF THE FIGURES

FIG. 1 illustrates the one-way analysis of pain VAS scores over the course of time post injection from patients administered 250 mg mirikizumab and 4000 U of rHuPH20 in a 2 mL dose.

FIG. 2 illustrates the one-way analysis of pain VAS scores over the course of time post injection from patients administered 250 mg mirikizumab in a 2 mL dose.

This invention is further illustrated by the following non-limiting examples.

### EXAMPLES

### Example 1: Clinical Study 1

### Overview

The addition of a hyaluronidase enzyme to mirikizumab drug product was evaluated to determine if it would enable the SC administration of larger volumes and if it would impact the tolerability of the drug product, including injection site pain. The impact on bioavailability of mirikizumab was also determined. The study determined the absolute bioavailability of mirikizumab following SC doses of mirikizumab co-administered with rHuPH20 compared to a 1200 mg mirikizumab intravenous (IV) dose in healthy subjects.

### Objectives

The study objectives are as follows:
(i) To assess the safety and tolerability of mirikizumab co-administered with rHuPH20 and mirikizumab in healthy subjects, including assessment of injection site pain (VAS); and
(ii) To evaluate the absolute bioavailability of mirikizumab following SC doses of mirikizumab co-administered with rHuPH20 in healthy subjects.

### Study Design

This was a single-center, randomized, open-label, Phase 1, single-dose study. Healthy male and female subjects aged 18 to 65 years, inclusive, with a body mass index of 18.0 to 32.0 kg/m2, inclusive, at the time of screening were eligible to be enrolled into this study. In a treatment arm, 36 subjects were enrolled as shown in Table 1:

TABLE 1
Treatment Assignments

| | Number of | |
|---|---|---|
| Group | Subjects | Treatment |
| 1 | 18 | 1200 mg mirikizumab IV dose |
| | 18 | 250 mg mirikizumab co-administered with 4000 U |
| | | rHuPH20 |

Safety and tolerability data collected up to Day 15 from all subjects administered the 250 mg mirikizumab co-administered with 4000 U rHuPH20 SC dose in Group 1 were reviewed.

Subjects were evaluated for study eligibility ≤28 days prior to enrollment and reported to the clinical research unit (CRU) on Day −1. Following randomization, subjects were administered study drug by either IV infusion or SC injection/infusion on the morning of Day 1. Subjects remained at the CRU until Day 2 after the completion of infusion/injection-site assessments and collection of pharmacokinetic (PK) blood samples for determination of concentrations of mirikizumab. Subjects returned to the unit for outpatient visits on Day 4 (±1 day), 8 (±1 day), 11 (11 day; for subjects administered mirikizumab+PH20 by SC injection/infusion only), 15 (±2 days), 22 (±2 days), 29 (±2 days), 43 (±2 days), 57 (±3 days), 71 (±3 days), and 85 (±3 days) for collection of PK blood samples and infusion/injection-site assessments.

Safety assessments performed during the study included the recording of adverse events (AEs), clinical laboratory evaluations, vital signs, electrocardiograms (ECGs), physical examinations/medical assessments, immunogenicity, and injection/infusion-site assessments.

Healthy subjects are frequently used in the assessment of bioavailability of small and large molecules, and mitigate the potential confounding effect of the disease state and concomitant medications in patients. Single doses of mirikizumab and mirikizumab co-administered with rHuPH20, and the PK sampling time points were selected to generate PK profiles sufficient to fulfill the study objectives. As the primary endpoints of this study are PK-related and were considered objective in nature, it was not considered appropriate to include a placebo control during this study.

### Study Drug Formulation and Administration

Mirikizumab was supplied for extemporaneous preparation at the site as a frozen solution at a concentration of 125 mg/mL, with approved excipients added. The solution was thawed at the site, pooled, and underwent terminal sterile filtration into sterile vials before being dispensed in syringes for SC injection/infusion, or further diluted for IV administration.

rHuPH20 was supplied as a sterile solution in glass vials, containing 0.5 mL rHuPH20 solution and stored at approximately −20° C.

For the co-administration treatments, a single dose was prepared by combining 2000 units rHuPH20 per mL of mirikizumab. For the 250 mg mirikizumab+rHuPH20 SC dose, an initial dilution of the rHuPH20 solution was made prior to rHuPH20 being combined with mirikizumab.

The treatments administered in the study are summarized in Table 2.

TABLE 2
Treatments administered

| Treatment Regimen | | |
|---|---|---|
| | 250 mg | 1200 mg |
| | mirikizumab + | mirikizumab |
| | rHuPH20 SC | IV |

| Approx. | 2.0 | mL | 40 | mL |
|---|---|---|---|---|
| volume | | | | |
| mirikizumab | | | | |
| per dose | | | | |
| Units of | 4000 | U | N/A | |
| rHuPH20 per | | | | |
| dose | | | | |
| Total volume | 2.0 | mL | 40 | mL |
| of dose | | | | |
| Final | 123 | mg/mL | 30 | mg/mL |
| concentration | | | | |
| of | | | | |
| mirikizumab | | | | |
| per dose | | | | |

| | | | |
|---|---|---|---|
| Duration of | Approx. 10 | ≥30 | minutes |
| injection | seconds | | |
| infusion | | | |

### Evaluation Methods

### a) Pain VAS

The pain VAS is a participant administered single item scale designed to measure pain using a 0-100 millimeter (mm) horizontal VAS. Overall severity of participant's pain is indicating by placing a single mark on the horizontal 100 mm scale from 0 mm (no pain) to 100 mm (worst imaginable pain). Assessments of injection pain intensity were performed within 1 minute (TO), 30 min, 1 hour, 3 hours, 6 hours and 24 hours following SC injection using the VAS.

### b) Bioanalytical Methods

Human serum samples obtained during this study were analyzed at ICON Laboratory Services, Inc. located in Whitesboro, N.Y., USA. The samples were analyzed for mirikizumab using a validated enzyme-linked immunosorbent assay (ELISA) method. The lower limit of quantification was 100 ng/mL, and the upper limit of quantification was 10,000 ng/mL. The inter-assay precision (% relative coefficient of variation) during validation ranged from 2.65% to 7.65%. Mirikizumab was stable for up to 1018 days when stored at approximately −70° C.

### c) Pharmacokinetic Analyses

Venous blood samples of approximately 2 mL each for the measurement of serum concentrations of mirikizumab were collected at the following times: 0 hours (predose; within 15 minutes of dosing) and at the end of infusion (IV treatment only), and 2 hours, 6 hours, 24 hours, 3 days, 7 days, 10 days, 14 days, 21 days, 28 days, 42 days, 56 days, 70 days, and 84 days hours post-dose. The time allowances for the 2, 6, and 24 hour time points were ±15, ±30, and ±90 minutes, respectively.

Pharmacokinetic parameters shown were determined from serum concentrations of mirikizumab using non-compartmental procedures with a validated software program (Phoenix WinNonlin Version 6.4). The primary parameters for analysis were area under the concentration versus time curve (AUC) from time zero to infinity (AUC[0-∞]) and AUC from time zero to time t, where t is the last time point with a measurable concentration (AUC[0-tlast]). The secondary PK parameters for analysis were maximum observed drug concentration (Cmax) and time of Cmax (tmax).

Concentrations at a sampling time that exceeded the protocol-specified time window were included in the mean concentration profiles. Actual sampling times were used in the PK analyses, with the exception of pre-dose sampling times, which were set to zero.

### d) Mirikizumab Immunogenicity

Serum samples obtained during this study were analyzed at PBI located in Seattle, Wash., USA. Samples were analyzed using a validated affinity capture elution ELISA to screen for, confirm, titer, and test for neutralizing antidrug antibody (ADA) against mirikizumab. The sensitivity of the screening ADA assay is 28.3 ng/mL with a drug tolerance of >500 μg/mL in the presence of 500 ng/mL affinity purified hyper-immunized monkey anti-mirikizumab antibody.

A treatment-emergent ADA (TE ADA) was defined as: having a negative ADA at baseline and an ADA titer greater than or equal to 1:20 (i.e., greater than 2-fold from the minimal required dilution of 1:10) any time post-baseline (i.e., treatment-induced); or a 4-fold or greater change in ADA titer from baseline for subjects that had a detectable ADA titer at baseline (i.e., treatment boosted). Antibodies were further characterized for their ability to neutralize the activity of the investigational product.

e) rHuPH20 Immunogenicity

Plasma samples were sent to Eurofins located in St. Charles, Mo., USA. Samples were analyzed in a bridging electrochemiluminescent screening assay that had a sensitivity of 15.6 ng/mL and drug tolerance of 1024 ng/mL of rHuPH20 in the presence of 250 ng/mL rabbit anti-rHuPH20 polyclonal antibody. Samples confirmed as positive in the screening rHuPH20 ADA assay were tested for neutralizing capacity at MicroConstants Inc, San Diego, Calif., USA. The assay is a colorimetric hyaluronidase enzymatic assay with a sensitivity of 52.4 μg/mL for immunoglobulin G affinity purified against rHuPH20. Full details are presented in the individual validation reports, which are available upon request.

Treatment-emergent ADA was defined as: having a negative ADA at baseline and an ADA titer greater than or equal to 1:10 (i.e., greater than 2-fold from the minimal required dilution of 1:5) any time post-baseline (i.e., treatment-induced); or a 4-fold or greater change in ADA titer from baseline for subjects that had a detectable ADA titer at baseline (i.e., treatment-boosted).

### Statistical Methods

### a) Pharmacokinetic Statistical Methods

The PK parameter estimates were analyzed to assess the absolute bioavailability of mirikizumab following a SC dose of mirikizumab+rHuPH20 compared to a 1200 mg mirikizumab IV dose.

Log-transformed dose normalized (DN)-AUC(0-∞) and DN-AUC(0-tlast) estimates were evaluated in a linear fixed-effects model with a fixed effect for treatment. The differences between the SC dose compared to the IV dose were back-transformed to present the ratios of geometric least squares (LS) means and the corresponding 90% confidence intervals (CIs).

The PK parameter estimates following SC administration were analyzed using a power model to assess the relative bioavailability between the SC treatments. Log-transformed AUC(0-∞), AUC(0-tlast), and Cmax estimates were evaluated in a power model with log-transformed dose as the explanatory variable. The treatment differences were back-transformed to present the ratios of geometric means and the corresponding 90% CIs. Whether injection/infusion-site leakage occurred was also included as a covariate in the models.

The tₘₐₓ was analyzed using a Wilcoxon rank sum test for each of the above comparisons.

Estimates of the median difference, 90% CIs, and p-values from the Wilcoxon rank sum test were calculated.

### b) Statistical Evaluation of Safety

Safety parameters were listed and summarized using descriptive statistics. All AEs were listed. Treatment-emergent AEs (TEAEs) were summarized by treatment, severity, and relationship to the study drug. The frequency of treatment-emergent AEs (absolute number of AEs, and number and percentage of subjects experiencing an AE) were summarized by treatment, Medical Dictionary for Regulatory Activities (MedDRA) Version 19.1 system organ class, and MedDRA preferred term. The summary and frequency AE tables were presented for all causalities and those considered related to the study drug. Any serious AEs (SAEs) were tabulated. Concomitant medication was coded using the World Health Organization (WHO) drug dictionary (Version September 2016). Injection-/Infusion-site assessment data were listed and summarized using descriptive statistics.

Injection-site pain was assessed using the visual analog scale (VAS). The average VAS scores (arithmetic means) were calculated for each treatment group. Injection/infusion-site leakage data and any bleeding were listed for subjects receiving SC doses.

Immunogenicity data were listed and frequency tables were presented for mirikizumab and rHuPH20. The frequency of TE ADAs was also calculated, and any associations between TE ADAs and AEs summarized by MedDRA preferred term.

### Results: Pain Visual Analog Score

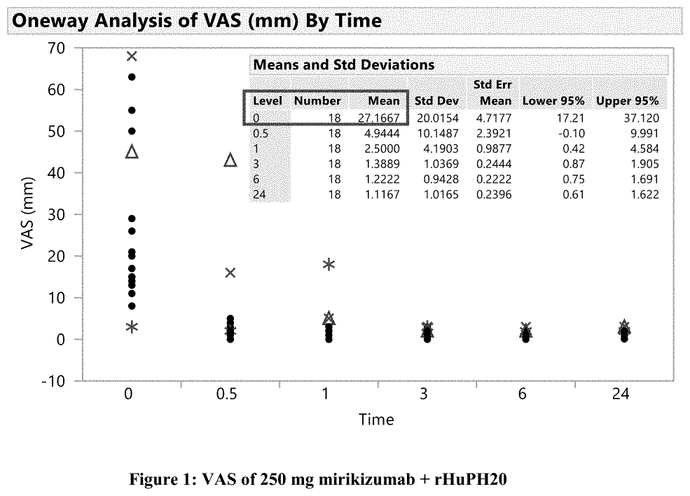

The results of VAS data over the course of time post injection are shown in FIG. 1. The time 0 injection pain intensity VAS score is determined to have an arithmetic mean of 27 mm.

### Results: Immunogenicity

### a) Mirikizumab

The overall presence of TE ADA to mirikizumab was higher following SC administration of 250 mg mirikizumab+rHuPH20 SC than following IV administration of 1200 mg mirikizumab.

Following administration 1200 mg mirikizumab IV, 3 (16.7%) subjects had TE ADA to mirikizumab at Day 85. Eighteen subjects (33%) had TE ADA to mirikizumab at ≥1 time point following administration of 250 mg mirikizumab+rHuPH20 SC.

Titers for mirikizumab ranged from 1:10 to 1:80 in the majority of subjects with ADA detected, and reached a maximum of 1:160 in 3 subjects and 1:320 in 2 subjects. The majority of subjects with ADA to mirikizumab detected also had neutralizing ADA detected, with between 7 and 11 subjects (38.9% to 61.1%) in each treatment group with neutralizing ADA detected.

There was no association between TE ADA to mirikizumab and TEAEs, TE ADA to mirikizumab and CL (IV dose) or CL/F (SC dose), or neutralizing antibodies to mirikizumab and CL (IV dose) or CL/F (SC doses) in this study. Additionally, there was no clear association between neutralizing antibodies and titers.

b) rHuPH20

No TE ADA to rHuPH20 were observed following SC administration of 250 mg mirikizumab+rHuPH20 SC. One subject (5.6%) had TE ADA to rHuPH20 on Days 29 and 85 following IV administration of 1200 mg mirikizumab. This subject was not administered mirikizumab+rHuPH20 SC or rHuPH20.

Titers for rHuPH20 ranged from 1:5 to 1:40 in the majority of subjects with ADA detected, but reached a maximum of 1:80 in 1 subject and 1:640 in 1 subject. The subject that had a titer of 1:640 had a predose titer of 1:320 and did not meet the criteria for TE ADA.

No subjects had neutralizing ADA to rHuPH20 detected.

### Results: Pharmacokinetics and Pharmacodynamics

The geometric mean (90% CI) absolute bioavailability of mirikizumab following 250 mg mirikizumab+rHuPH20 SC doses was 0.447 (0.370, 0.539) when assessed by DN-AUC(0-∞).

### Example 2: Clinical Study 2

### Overview

This study was designed to determine the pain VAS, pharmacokinetics (PK) and tolerability of mirikizumab when administered SC to healthy subjects. The potential effect of administering the same volume as a single injection (1×2 mL) or as two injections (2×1 mL) on pain VAS, PK and tolerability was also be investigated during SC administration. In addition, the absolute bioavailability of mirikizumab was investigated by inclusion of an IV mirikizumab treatment.

### Objectives

The study objectives were as follows:
(i) To assess the impact of number of injections on tolerability of mirikizumab, including assessment of injection site pain (VAS);
(ii) To determine the relative bioavailability of the mirikizumab lyophilized formulation prepared by reconstitution and the extemporaneously prepared test formulation (SC 2×1 mL) when administered to healthy subjects through SC injection;
(iii) To determine relative bioavailability of mirikizumab extemporaneously prepared as the test formulation when the same volume is administered to healthy subjects through a single SC injection (1×2 mL) and as two SC injections (2×1 mL); and
(iv) To determine the absolute bioavailability of mirikizumab extemporaneously prepared as the test formulation when administered to healthy subjects through SC injection compared to IV infusion.

### Study Design

This study was a single-center, open-label, randomized, parallel treatment, single-dose administration Phase 1 study evaluating mirikizumab in up to 72 healthy subjects. Subjects were evaluated for study eligibility ≤28 days prior to enrollment. Subjects who fulfilled the eligibility criteria were randomized to 1 of 4 treatments, with up to 18 subjects randomized to each treatment, as shown below:
i) 250 mg mirikizumab lyophilized reference formulation with 2×1-mL+1×1.5-mL SC injections;
ii) SC 2×1-mL: 250 mg mirikizumab test formulation with 2×1-mL SC injections;
iii) SC 1×2-mL: 250 mg mirikizumab test formulation with 1×2-mL SC injection;
iv) 250 mg mirikizumab formulation through IV infusion over at least 30 minutes.

Other than the absence of a hyaluronidase enzyme, the test formulations referred to in ii) and iii) hereinabove are the same as the mirikizumab+rHuPH20 formulation referred to in Example 1.

Subjects reported to the clinical research unit (CRU) on Day −1 and remained at the CRU until after all the scheduled procedures had been completed on Day 2. Study drug was administered through either IV infusion or SC injection according to the randomization schedule, in the morning of Day 1 after an overnight fast. The follow-up period included outpatient visits for a total of 12 weeks following Day 1 dose administration to assess tolerability and PK of mirikizumab. An end-of-study follow-up visit was also scheduled within 7 to 14 days after the last procedure or upon early discontinuation.

Single doses of 250 mg mirikizumab and the PK sampling time points were selected to generate PK profiles sufficient to fulfill the study objectives. Since the bioavailability endpoints were PK related and therefore considered to be objective in nature, it was not considered appropriate to include a placebo control during this study. Furthermore, the objective relating to tolerability was a comparison between formulations and therefore addition of a placebo control did not contribute to the scientific validity of the study.

As only a single 250-mg dose of mirikizumab was administered during this study, and subjects and site staff were aware of the administration route (number of SC injections, IV dosing), an unblinded study design was selected.

As the maximum achievable concentration of the lyophilized reference formulation is 72 mg/mL, it is not possible to administer 250 mg with a total injection volume of 2 mL for consistency with the other SC treatments. The total injection volume for the reference formulation was therefore increased to 3.5 mL (2×1 mL+1×1.5 mL, three SC injections) to ensure a comparable total dose to the test formulation. Although the theoretical total dose for this treatment is 252 mg, the dose levels for all treatments are referred to as 250 mg for the purposes of this study.

A parallel-group design was chosen because a crossover design is impractical for compounds that have long half-lives, such as monoclonal antibodies. In addition, a crossover study design could confound PK data if subjects develop neutralizing antidrug antibodies (ADAs).

### Study Drug Formulation and Administration

The following treatments were administered:
a) Supplied as a frozen solution, the mirikizumab 250 mg test formulation was prepared extemporaneously at the CRU pharmacy and a single 250-mg dose was administered as either one SC injection (1×2 mL) or two SC injections (2×1 mL) or as an IV infusion over 30 minutes.
b) Mirikizumab 250-mg reference lyophilized formulation was supplied as a sterile lyophilized vial to be reconstituted at the CRU pharmacy and given as three SC injections (2×1 mL+1×1.5 mL).

Injection sites selected for SC administration were in the abdominal region approximately 5 cm from the umbilicus and the treatment administered with the needle applied at approximately 45 degrees with pinching of the skin. The same type of syringe and needle (27-gauge, half-inch needle) was used for all subjects to ensure all injections are delivered to a consistent depth target into the SC space. Where two or more injections were administered, the subsequent injection(s) were given immediately following the previous injection and administered to another abdominal quadrant.

The IV formulation of LY3074828 was administered as a slow IV infusion over at least 30 minutes.

The treatments administered in the study are summarized in Table 3.

TABLE 3
Treatments administered

| | Treatment Regimen | | | |
|---|---|---|---|---|
| | | 250 mg | 250 mg | |
| | 250^{a} mg | mirikizumab SC | mirikizumab SC | 250 mg |
| | mirikizumab SC | 2 × 1 mL | 1 × 2 mL | mirikizumab IV |
| | (N = 18) | (N = 18) | (N = 18) | (N = 18) |
| Drug Product | From lyophilized | From frozen | From frozen | From frozen |
| formulation | supply | solution | solution | solution |
| Mirikizumab | 72 mg/mL | 125 mg/mL | 125 mg/mL | 250 mg/10 mL |
| concentration | | | | (25 mg/mL) |
| Total volume | 2 × 1 mL + 1 × 1.5 mL | 2 × 1 mL | 1 × 2 mL | 10 mL infusion |
| of dose | (3.5 mL total) | (2 mL total) | (2 mL total) | |
| Duration of | N/A | N/A | Slow versus fast | ≥30 minutes |
| injection | | | | |
| infusion | | | | |

^{a}Theoretical dose of 252 mg
b Odd number of subjects will be injected slowly, approximately over 15 seconds and even number of subjects will receive the injection over 5 to 10 seconds

### Evaluation Methods

### a) Pain VAS

The pain VAS is a participant administered single item scale designed to measure pain using a 0-100 millimeter (mm) horizontal VAS. Overall severity of participant's pain is indicating by placing a single mark on the horizontal 100 mm scale from 0 mm (no pain) to 100 mm (worst imaginable pain). Assessments of injection pain intensity were performed within 1 minute (TO), 30 min, 1 hour, 3 hours, 6 hours and 24 hours following SC injection using visual analog scale (VAS).

### b) Bioanalytical Methods

Serum samples obtained during this study were analyzed for mirikizumab using a validated enzyme-linked immunosorbent assay (ELISA) method at ICON Laboratory Services, Inc. located in Whitesboro, N.Y., USA. The lower limit of quantification was 100 ng/mL, and the upper limit of quantification was 10,000 ng/mL. Samples above the limit of quantification were diluted to yield results within the calibrated range. The inter-assay accuracy (% relative error) during validation ranged from −9.09% to 2.34%. The inter-assay precision (% coefficient of variation) during validation ranged from 2.65% to 7.65%. Mirikizumab was stable for up to 1018 days when stored at approximately −70° C.

### c) Pharmacokinetic Analysis

Venous blood samples were collected to determine the serum concentrations of mirikizumab at 0, end of infusion, 2 hours, 6 hours, 24 hours, 72 hours, 168 hours, 240 hours, 336 hours, 504 hours, 672 hours, 1008 hours, 1344 hours, 1680 hours and 2016 hours. The times are referenced to start of dosing. 0 hour collection should be taken immediately (within 15 minutes) before dosing. The end of infusion sampling should be taken at the end of IV infusion. Time allowance for 2, 6, and 24 hour time points will be ±15, ±30, and ±90 minutes respectively.

Pharmacokinetic parameters shown in Table 4 were determined from serum concentrations of mirikizumab using non-compartmental procedures with a validated software program (Phoenix WinNonlin Version 6.4).

TABLE 4
Pharmacokinetic parameters

| Parameter | Definition |
|---|---|
| AUC(0-tₗₐₛₜ) | area under the concentration versus time curve from time |
| | zero to time t, where t is the last time point with a |
| | measurable concentration |
| AUC(0-∞) | area under the concentration versus time curve from |
| | time zero to infinity |
| %AUC(tₗₐₛₜ-∞) | percentage of AUC(0-∞) extrapolated |
| Cₘₐₓ | maximum observed drug concentration |
| tₘₐₓ | time of maximum observed drug concentration |
| t_{1/2} | half-life associated with the terminal rate constant is |
| | noncompartmental analysis |
| CL/F | apparent total body clearance of drug calculated after |
| | extravascular administration |
| V_{Z}/F | apparent volume of distribution during the terminal phase |
| | after extravascular administration |
| V_{SS}/F | apparent volume of distribution at steady state after |
| | extravascular administration |
| CL | total body clearance of drug calculated after IV |
| | administration |
| V_{Z} | volume of distribution during the terminal phase after |
| | IV administration |
| V_{SS} | volume of distribution at steady state after IV |
| | administration |
| F | absolute subcutaneous bioavailability |
| Frel | relative bioavailability |

Actual sampling times were used in the PK analyses, with the exception of pre-dose sampling times which were set to zero. Concentrations at a sampling time exceeding ±10% were excluded from the average concentration profiles. Half-life estimated over a time window of less than 2 half-lives was flagged in the data listings.

Subjects with injection-site leakage were flagged in the PK parameter data listings.

### d) Immunogenicity

Serum samples were collected at Days 0, 15, 29 and 85 were analyzed at PBI located in Seattle, Wash., USA. Samples were analyzed using a validated affinity capture elution ELISA to screen for, confirm, titer, and test for neutralizing ADA against mirikizumab. The sensitivity of the screening ADA assay is 28.3 ng/mL with a drug tolerance of >500 μg/mL in the presence of 500 ng/mL affinity purified hyper-immunized monkey anti-mirikizumab antibody.

A treatment-emergent ADA (TE-ADA) was defined as having a negative ADA at baseline and an ADA titer greater than or equal to 1:20 (i.e., greater than 2-fold from the minimal required dilution of 1:10) any time post-baseline (i.e., treatment-induced); or a 4-fold or greater change in ADA titer from baseline for subjects that had a detectable ADA titer at baseline (i.e., treatment boosted). Antibodies were further characterized for their ability to neutralize the activity of mirikizumab.

### Statistical Methods

### a) Pharmacokinetic Statistical Methodology

Log-transformed maximum observed drug concentration (Cmax), area under the concentration versus time curve (AUC) from time zero to infinity (AUC[0-∞]), and AUC from time zero to time t, where t is the last time point with a measurable concentration (AUC[0-tlast]) estimates were evaluated in a linear fixed-effect model with a fixed effect for formulation. Whether injection-site leakage occurred (yes/no) was also included in the model as a covariate if leakage occurred frequently. Furthermore, if this was found to be significant, the amount of leakage was also added to the model. The formulation differences were back transformed to present the ratios of geometric least squares (LS) means and the corresponding 90% confidence interval (CI).

A single linear model with contrasts was used for all of the following comparisons:
Relative bioavailability of formulations: Test Formulation SC 2×1 mL versus Lyophilized Formulation
Relative bioavailability of number of injections: Test Formulation SC 2×1 mL versus Test Formulation SC 1×2 Ml

As the number of injections was observed to have an effect, analyses to evaluate absolute bioavailability were done separately:
Test Formulation SC 1×2 mL versus Test Formulation IV
Test Formulation SC 2×1 mL versus Test Formulation IV

The primary analysis of absolute bioavailability was considered as that including SC 1×2 mL.

The time of maximum observed drug concentration (tmax) was analyzed using a Wilcoxon rank sum test for the relative bioavailability comparisons. Estimates of the median difference, 90% CIs, and p-values from the Wilcoxon rank sum test were calculated. Additional analyses may have been conducted if they were deemed appropriate.

### b) Statistical Evaluation of Safety and Tolerability

Where changes in severity were recorded in the case report form, each separate severity of the adverse event (AE) was reported in the listings; only the most severe was used in the summary tables. A pre-existing condition is defined as an AE that starts before the subject has provided written informed consent and is ongoing at consent. A non-treatment-emergent adverse event (TEAE) is defined as an AE that starts after informed consent but prior to dosing. A TEAE is defined as an AE that occurs post-dose or that is present prior to dosing and becomes more severe post-dose.

All AEs were listed. Treatment-emergent adverse events were summarized by treatment, severity, and relationship to the study drug. The frequency (the number of AEs, the number of subjects experiencing an AE, and the percentage of subjects experiencing an AE) of TEAEs was summarized by treatment, Medical Dictionary for Regulatory Activities version 19.1 system organ class and preferred term. The summary and frequency of AE tables were presented for all causalities and those considered related to the study drug. Any serious AEs were tabulated.

Solicited findings at the injection site, e.g., erythema, swelling, or pain, were recorded on the injection-site reaction (ISR) form and any positive findings were additionally recorded as an AE item.

Injection/infusion-site assessment data (erythema, induration, categorical pain, pruritus, and edema) are listed.

Injection-site pain was assessed using the visual analog scale (VAS). The average VAS scores (arithmetic means) were calculated for each treatment group. In addition, categorical grouping of the VAS data was performed to link VAS data to clinical relevance, per following criteria (0-30 mm mild pain; 31-70 mm moderate pain; 71-100 mm severe pain).

Injection-site leakage data were listed. Any bleeding from injection site was also documented (performed only on subjects randomized to Test Formulation SC 2×1 mL and Test Formulation SC 1×2 mL).

### Results: Pain VAS

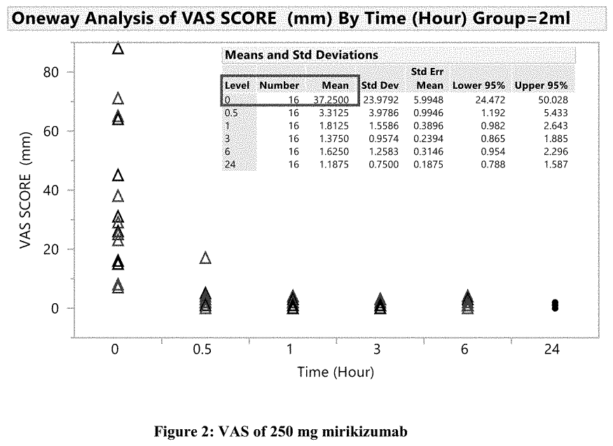

The results of VAS data over the course of time post injection are shown in FIG. 2. The time 0 injection pain intensity VAS score is determined to have an arithmetic mean of 37 mm.

### Results: Safety and Tolerability

Of the 67 subjects who received mirikizumab, 54 reported AEs that were related to mirikizumab as judged by the investigator. The most common AEs related to study treatment were ISRs (reported by 49 of 50 subjects who received SC dose administration) and injection-site bruising. The high incidence of ISRs may be attributable to the method of reporting.

Any positive findings solicited during the assessment, including barely visible erythema or injection pain, which were otherwise asymptomatic, were recorded as ISR AE items per protocol. Most AEs were of mild severity.

The frequency of injection site reactions following SC administration was similar across the three SC treatment groups. Twenty-two out of 50 subjects (44%) reported “well-defined erythema” with the majority of subjects reporting an occurrence either immediately after (0 hour) the injection or 15-minute post-dose. As only two subjects reported “well-defined erythema” following administration of the Lyophilized Formulation at 1 and 14 days post-dose, a majority of ISRs due to erythema were considered transient.

A majority of ISRs for pain were rated as mild in severity with similar incidence across all SC treatment groups.

For each formulation, the ISRs showed no induration.

### Results: Pharmacokinetics and Pharmacodynamics

A total of 67 healthy subjects, 39 male and 28 female, between the ages of 19 to 65 years participated in this study. All of the 67 subjects who gave consent and were enrolled into the study were successfully dosed with mirikizumab according to the randomization schedule.

Data from 67 subjects who received mirikizumab were included in the PK analysis. Three subjects in the Test Formulation SC 2×1-mL group had a quantifiable pre-dose concentration that was excluded from PK analysis and the mean concentration versus time profile.

One subject in the Test IV group withdrew from the study following the Day 43 (1008 hours post-dose) PK blood sample. As a result, AUC(0-tlast) was flagged and excluded from the descriptive statistics and statistical analysis.

Injection-site leakage was reported for 20 subjects following SC administration and all subjects were included in descriptive statistics and statistical analysis.

The mean serum mirikizumab concentration versus time profiles following single SC injection of Lyophilized Formulation and Test Formulation SC 1×2 mL were similar.

Systemic exposure of mirikizumab following Test Formulation SC 2×1-mL treatment was shown to be similar to the Lyophilized SC treatment. Statistical analysis showed that the ratios of the geometric LS mean for AUC(0-tlast), AUC(0-∞), and Cmax were 0.996, 0.996, and 0.986, respectively, and the 90% CIs included unity for all parameters. The median tₘₐₓ was similar between the 2 treatments, with no statistically significant difference observed (p=0.368).

Following administration of the extemporaneously prepared Test Formulation as 2 SC injections (Test SC 2×1 mL) compared to a single SC injection (Test SC 1×2 mL), systemic exposure of mirikizumab based on AUC(0-tlast), AUC(0-∞), and Cmax was approximately 27%, 27%, and 25% lower, respectively, for Test SC 2×1 mL. The 90% CIs for the ratios of geometric LS means excluded unity for all parameters, indicating that the differences were statistically significant. The tₘₐₓ was not statistically significantly different following administration of Test Formulation SC 2×1 mL compared with Test Formulation SC 1×2 mL (p=0.358).

Between-subject variability estimates for AUC(0-∞) and Cmax were moderate (31% to 36% and 32% to 34%, respectively) following administration of Lyophilized Formulation SC and Test Formulation SC 1×2 mL.

Higher variability was observed for Test Formulation SC 2×1 mL compared to the other treatments with geometric coefficient of variation (CV %) of 55% for AUC(0-∞) and 51% for Cmax.

The statistical analysis indicated that the absolute SC bioavailability of mirikizumab based on AUC(0-∞) was 42% following Test Formulation SC 1×2 mL administration and 31% following Test Formulation SC 2×1 mL administration versus Test IV.

Between-subject variability estimates were low following IV administration with geometric CV % for AUC(0-∞) and Cmax being 14% and 22%, respectively.

Overall, based on mirikizumab AUC(0-tlast), AUC(0-∞), and Cmax, the Test Formulation SC 2×1 mL was found to be similar to the Lyophilized Formulation SC with the ratios of the geometric LS mean close to unity and 90% C is were contained within the traditional bioequivalence boundary limits of 0.80 to 1.25. There were no statistically significant differences in tₘₐₓ between formulations.

When the same volume of the extemporaneously prepared formulation was administered using a single SC injection (Test Formulation SC 1×2 mL) compared to two SC injections (Test Formulation SC 2×1 mL), the relative bioavailability based on AUC (AUC[0-tlast] and AUC[0-∞]) and Cmax was approximately 73%, and 75%, respectively. There were no statistically significant differences in tₘₐₓ between the treatments.

The absolute bioavailability of mirikzumab following SC administration of Test Formulation SC 1×2 mL or Test Formulation SC 2×1 mL relative to IV dosing was 42% and 31%, respectively.

### Results: Comparison of VAS Data Across Clinical Studies

The comparison of VAS data shown in FIG. 1 and FIG. 2 indicate the mean VAS for 18 subjects administered with 2 mL mirikizumab (250 mg dose) and hyaluronidase is 27 mm, which is significantly less than that (Mean 37 mm) for 2 mL injection of mirikizumab (250 mg dose) alone without hyaluronidase. This difference of 10 mm in VAS score suggest there is a clinically significant reduction in injection pain intensity after introducing hyaluronidase to mirikizumab solution.

An alternative way of assessing injection pain intensity for clinical relevance is categorical grouping of VAS data, based on the following criteria: mild pain (0-30 mm VAS), moderate pain (31-70 mm VAS) and severe pain (71-100 mm VAS). Table 5 shows the comparison of categorical grouping of injection pain for 250 mg mirikizumab alone or with a hyaluronidase enzyme. The data indicate that the hyaluronidase enzyme reduces the levels of moderate pain (from 37.5% to 27.8%) or severe pain (12.5% to 0%) in 2 mL injection of mirikizumab.

TABLE 5
Categorical grouping of injection pain for 250 mg
mirikizumab alone or with a hyaluronidase enzyme

| | | Pain Intensity Category (VAS) | | | |
|---|---|---|---|---|---|
| | | Mild | Moderate | Severe | |
| Study | | (0-30 | (31-70 | (71-100 | |
| Volume | Dose/Drug | mm) | mm) | mm) | N |
| Study 1: | 250 mg mirikizumab + | 72.2% | 27.8% | 0 | 18 |
| 2 mL | rHuPH20 | | | | |
| Study 2: | 250 mg mirikizumab | 50.0% | 37.5% | 12.5% | 16 |
| 2 mL | | | | | |

### NUMBERED EMBODIMENTS

1. A method of treating an IL-23 related disorder wherein the method comprises administering in simultaneous, separate or sequential combination to a patient in need thereof, effective amounts of mirikizumab and a hyaluronidase enzyme by subcutaneous injection.
2. A method of treating an IL-23 related disorder according to numbered embodiment 1, wherein the hyaluronidase enzyme is recombinant human hyaluronidase enzyme (CAS-Registry No. 757971-58-7).
3. A method of treating an IL-23 related disorder according to numbered embodiment 1 or numbered embodiment 2, wherein the effective amount of mirikizumab is 50-5000 mg.
4. A method of treating an IL-23 related disorder according to any one of numbered embodiments 1-3, wherein the effective amount of mirikizumab is 50 mg, 100 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg.
5. A method of treating an IL-23 related disorder according to any one of numbered embodiments 1-4, wherein the effective amount of mirikizumab is 100 mg.
6. A method of treating an IL-23 related disorder according to any one of numbered embodiments 1-4, wherein the effective amount of mirikizumab is 125 mg.
7. A method of treating an IL-23 related disorder according to any one of numbered embodiments 1-4, wherein the effective amount of mirikizumab is 200 mg.
8. A method of treating an IL-23 related disorder according to any one of numbered embodiments 1-4, wherein the effective amount of mirikizumab is 250 mg.
9. A method of treating an IL-23 related disorder according to any one of numbered embodiments 1-4, wherein the effective amount of mirikizumab is 600 mg.
10. A method of treating an IL-23 related disorder according to any one of numbered embodiments 1-4, wherein the effective amount of mirikizumab is 1000 mg.
11. A method of treating an IL-23 related disorder according to any one of numbered embodiments 1-4, wherein the effective amount of mirikizumab is 1200 mg.
12. A method of treating an IL-23 related disorder according to any one of numbered embodiments 1-4, wherein the effective amount of mirikizumab is 2400 mg.
13. A method of treating an IL-23 related disorder according to any one of numbered embodiments 1-12, wherein the effective amount of hyaluronidase enzyme is 1000-3000 U per mL of formulated injection solution.
14. A method of treating an IL-23 related disorder according to any one of numbered embodiments 1-13, wherein the effective amount of hyaluronidase enzyme is 1500-2500 U per mL of formulated injection solution.
15. A method of treating an IL-23 related disorder according to any one of numbered embodiments 1-14, wherein the effective amount of hyaluronidase enzyme is 2000 U per mL of formulated injection solution.
16. A method of treating an IL-23 related disorder according to any one of numbered embodiments 13-15, wherein the maximum effective amount of hyaluronidase enzyme is 50,000 U in 25 mL of formulated injection solution.
17. A method of treating an IL-23 related disorder according to any one of the preceding numbered embodiments, wherein the IL-23 related disorder is psoriasis, psoriatic arthritis, ulcerative colitis, Crohn's Disease or ankylosing spondylitis.
18. A method of treating an IL-23 related disorder according to any one of the preceding numbered embodiments, wherein mirikizumab and the hyaluronidase enzyme are co-formulated for simultaneous administration by subcutaneous injection.
19. Mirikizumab for use in the treatment of an IL-23 related disorder, wherein mirikizumab is administered in simultaneous, separate or sequential combination with a hyaluronidase enzyme and wherein administration of mirikizumab and the hyaluronidase enzyme is by subcutaneous injection.
20. Mirikizumab for use in the treatment of an IL-23 related disorder according to numbered embodiment 19, wherein the hyaluronidase enzyme is recombinant human hyaluronidase enzyme (CAS-Registry No. 757971-58-7).
21. Mirikizumab for use in the treatment of an IL-23 related disorder according to numbered embodiment 19 or numbered embodiment 20, wherein the amount of mirikizumab administered is 50-5000 mg.
22. Mirikizumab for use in the treatment of an IL-23 related disorder according to any one of numbered embodiments 19-21, wherein the amount of mirikizumab administered is 50 mg, 100 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg.
23. Mirikizumab for use in the treatment of an IL-23 related disorder according to any one of numbered embodiments 19-21, wherein the amount of mirikizumab administered is 100 mg.
24. Mirikizumab for use in the treatment of an IL-23 related disorder according to any one of numbered embodiments 19-21, wherein the amount of mirikizumab administered is 125 mg.
25. Mirikizumab for use in the treatment of an IL-23 related disorder according to any one of numbered embodiments 19-21 wherein the amount of mirikizumab administered is 200 mg.
26. Mirikizumab for use in the treatment of an IL-23 related disorder according to any one of numbered embodiments 19-21, wherein the amount of mirikizumab administered is 250 mg.
27. Mirikizumab for use in the treatment of an IL-23 related disorder according to any one of numbered embodiments 19-21, wherein the amount of mirikizumab administered is 600 mg.
28. Mirikizumab for use in the treatment of an IL-23 related disorder according to any one of numbered embodiments 19-21, wherein the amount of mirikizumab administered is 1000 mg.
29. Mirikizumab for use in the treatment of an IL-23 related disorder according to any one of numbered embodiments 19-21, wherein the effective amount of mirikizumab administered is 1200 mg.
30. Mirikizumab for use in the treatment of an IL-23 related disorder according to any one of numbered embodiments 19-21, wherein the effective amount of mirikizumab administered is 2400 mg.
31. Mirikizumab for use in the treatment of an IL-23 related disorder according to any one of numbered embodiments 19-30, wherein the amount of hyaluronidase enzyme administered is 1000-3000 U per mL of formulated injection solution.
32. Mirikizumab for use in the treatment of an IL-23 related disorder according to any one of numbered embodiments 19-31, wherein the amount of hyaluronidase enzyme administered is 1500-2500 U per mL of formulated injection solution.
33. Mirikizumab for use in the treatment of an IL-23 related disorder according to any one of numbered embodiments 19-32, wherein the amount of hyaluronidase enzyme administered is 2000 U per mL of formulated injection solution.
34. Mirikizumab for use in the treatment of an IL-23 related disorder according to any one of numbered embodiments 19-33, wherein the maximum amount of hyaluronidase enzyme is 50,000 U in 25 mL of formulated injection solution
35. Mirikizumab for use in the treatment of an IL-23 related disorder according to any one of the numbered embodiments 19-34, wherein the IL-23 related disorder is psoriasis, psoriatic arthritis, ulcerative colitis, Crohn's Disease or ankylosing spondylitis.
36. Mirikizumab for use in the treatment of an IL-23 related disorder according to any one of the numbered embodiments 19-35, wherein mirikizumab and the hyaluronidase enzyme are co-formulated for simultaneous administration by subcutaneous injection.
37. A combination comprising mirikizumab and a hyaluronidase enzyme, wherein the combination is administered by subcutaneous injection.
38. A combination according to numbered embodiment 37, wherein the hyaluronidase enzyme is recombinant human hyaluronidase enzyme (CAS-Registry No. 757971-58-7).
39. A combination according to numbered embodiment 37 or numbered embodiment 38 comprising 50-5000 mg of mirikizumab.
40. A combination according to any one of numbered embodiments 37-39 comprising 50 mg, 100 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg of mirikizumab.
41. A combination according to any one of numbered embodiments 37-39, comprising 100 mg of mirikizumab
42. A combination according to any one of numbered embodiments 37-39, comprising 125 mg of mirikizumab.
43. A combination according to any one of numbered embodiments 37-39, comprising 200 mg of mirikizumab.
44. A combination according to any one of numbered embodiments 37-39, comprising 250 mg of mirikizumab.
45. A combination according to any one of numbered embodiments 37-39, comprising 600 mg of mirikizumab.
46. A combination according to any one of numbered embodiments 37-39, comprising 100 mg of mirikizumab.
47. A combination according to any one of numbered embodiments 37-39, comprising 1200 mg of mirikizumab.
48. A combination according to any one of numbered embodiments 37-39, comprising 2400 mg of mirikizumab.
49. A combination according to any one of numbered embodiments 37-48, comprising 1000-3000 U hyaluronidase enzyme per mL of formulated injection solution.
50. A combination according to any one of numbered embodiments 37-49, comprising 1500-2500 U of hyaluronidase enzyme per mL of formulated injection solution.
51. A combination according to any one of numbered embodiments 37-50, comprising 2000 U of hyaluronidase enzyme per mL of formulated injection solution.
52. A combination according to any one of numbered embodiments 37-50, comprising a maximum of 50,000 U of hyaluronidase enzyme in 25 mL of formulated injection solution.
53. A pharmaceutical formulation for subcutaneous injection comprising 100-2400 mg of mirikizumab, and 1500-3000 U of recombinant human hyaluronidase enzyme (rHuPH20)(CAS-Registry No. 757971-58-7) per mL of formulated injection solution.
54. A pharmaceutical formulation according to numbered embodiment 53, wherein the formulation comprises 100-2400 mg of mirikizumab, and 2000 U of rHuPH20 per mL of injection solution.
55. A pharmaceutical formulation according to numbered embodiment 53 or numbered embodiment 54, wherein the formulation comprises 100 mg of mirikizumab and 2000 U of rHuPH20 in 1 mL of injection solution.
56. A pharmaceutical formulation according to numbered embodiment 53 or numbered embodiment 54, wherein the formulation comprises 150 mg of mirikizumab and 2000 U of rHuPH20 in 1 mL of injection solution.
57. A pharmaceutical formulation according to numbered embodiment 53 or numbered embodiment 54, wherein the formulation comprises 200 mg of mirikizumab and 4000 U of rHuPH20 in 2 mL of injection solution.
58. A pharmaceutical formulation according to numbered embodiment 53 or numbered embodiment 54, wherein the formulation comprises 250 mg of mirikizumab and 4000 U of rHuPH20 in 2 mL of injection solution.
59. A pharmaceutical formulation according to numbered embodiment 53 or numbered embodiment 54, wherein the formulation comprises 300 mg of mirikizumab and 4000 U of rHuPH20 in 2 mL of injection solution.
60. A pharmaceutical formulation according to numbered embodiment 53 or numbered embodiment 54, wherein the formulation comprises 1200 mg of mirikizumab and 16,200 U of rHuPH20 in 8.1 mL of injection solution.
61. A pharmaceutical formulation according to numbered embodiment 53 or numbered embodiment 54, wherein the formulation comprises 2400 mg of mirikizumab and 39,200 U of rHuPH20 in 19.5 mL of injection solution.
62. A pharmaceutical formulation according to any one of numbered embodiments 53-61, wherein the formulation reduces the pain associated with subcutaneous injection compared to an equivalent formulation that does not comprise rHuPH20.
63. A kit comprising a first pharmaceutical formulation comprising 50-5000 mg of mirikizumab and a second pharmaceutical formulation comprising a recombinant human hyaluronidase enzyme (CAS-Registry No. 757971-58-7), wherein the kit further comprises instructions for combining the first and second formulations for co-administration such that the co-formulated formulation is suitable for subcutaneous injection and comprises 50-5000 mg of mirikizumab and 1000-3000 U of rHuPH20 per mL of injection solution.

1. A method of treating an IL-23 related disorder wherein the method comprises administering in simultaneous, separate or sequential combination to a patient in need thereof, effective amounts of mirikizumab and a hyaluronidase enzyme by subcutaneous injection.

2. A method of treating an IL-23 related disorder according to claim 1, wherein the hyaluronidase enzyme is recombinant human hyaluronidase enzyme (CAS-Registry No. 757971-58-7).

3. A method of treating an IL-23 related disorder according to claim 1, wherein the effective amount of mirikizumab is 50-5000 mg.

4. A method of treating an IL-23 related disorder according to claim 1, wherein the effective amount of mirikizumab is 50 mg, 100 mg, 125 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg.

5. A method of treating an IL-23 related disorder according to claim 4, wherein the effective amount of hyaluronidase enzyme is 1000-3000 U per mL of formulated injection solution.

6. A method of treating an IL-23 related disorder according to claim 5, wherein the effective amount of hyaluronidase enzyme is 1500-2500 U per mL of formulated injection solution.

7. A method of treating an IL-23 related disorder according to claim 6, wherein the IL-23 related disorder is psoriasis, psoriatic arthritis, ulcerative colitis, Crohn's Disease or ankylosing spondylitis.

8. A method of treating an IL-23 related disorder according to claim 6, wherein mirikizumab and the hyaluronidase enzyme are co-formulated for simultaneous administration by subcutaneous injection.

9. A combination comprising mirikizumab and a hyaluronidase enzyme, wherein the combination is administered by subcutaneous injection.

10. A combination according to claim 9, wherein the hyaluronidase enzyme is recombinant human hyaluronidase enzyme (CAS-Registry No. 757971-58-7).

11. A combination according to any one of claim 9 comprising 50 mg, 100 mg, 125 mg, 150 mg, 200 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300, 2400 mg, 2500 mg, 2600 mg, 2700 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg or 5000 mg of mirikizumab.

12. A combination according to claim 11, comprising 1000-3000 U hyaluronidase enzyme per mL of formulated injection solution.

13. A combination according to claim 12, comprising 1500-2500 U of hyaluronidase enzyme per mL of formulated injection solution.

14. A pharmaceutical formulation for subcutaneous injection comprising 100-2400 mg of mirikizumab, and 1500-3000 U of recombinant human hyaluronidase enzyme (rHuPH20)(CAS-Registry No. 757971-58-7) per mL of formulated injection solution.

15. A pharmaceutical formulation according to claim 14, wherein the formulation comprises 100-2400 mg of mirikizumab, and 2000 U of rHuPH20 per mL of injection solution.

16. A pharmaceutical formulation according to claim 14, wherein the formulation reduces the pain associated with subcutaneous injection compared to an equivalent formulation that does not comprise rHuPH20.

17. A kit comprising a first pharmaceutical formulation comprising 50-5000 mg of mirikizumab and a second pharmaceutical formulation comprising a recombinant human hyaluronidase enzyme (CAS-Registry No. 757971-58-7), wherein the kit further comprises instructions for combining the first and second formulations for co-administration such that the co-formulated formulation is suitable for subcutaneous injection and comprises 50-5000 mg of mirikizumab and 1000-3000 U of rHuPH20 per mL of injection solution.
